(19) United States
(12) Patent Application Publication
(10) Pub. No.: US 2021/0196931 A1
(43) Pub. Date: Jul. 1, 2021

(54) DELIVERY DEVICE FOR MEDICAL TUBULAR BODY

(71) Applicant: KANEKA CORPORATION, Osaka-shi, Osaka (JP)

(72) Inventor: Shizuo ICHIMURA, Settsu-shi (JP)

(73) Assignee: KANEKA CORPORATION, Osaka-shi, Osaka (JP)

(21) Appl. No.: 17/269,847

(22) PCT Filed: Apr. 23, 2019
(86) PCT No.: PCT/JP2019/017306

§ 371 Date: Feb. 19, 2021

(30) Foreign Application Priority Data
Aug. 24, 2018 (JP) 2018-157665

(51) Int. Cl. A61M 25/09; A61M 25/00; A61F 2/95

(52) U.S. Cl. CPC A61M 25/09041; A61M 2205/583; A61F 2/95; A61M 25/0045

(57) ABSTRACT

Disclosed is a delivery device for a medical tubular body that is unlikely to be kinked without decreasing handleability. The delivery device for a medical tubular body has an outer tube in which the medical tubular body is to be disposed; an inner rod disposed in the outer tube; and a guidewire insertion member disposed proximal to the medical tubular body, the guidewire insertion member having a penetration passage in which a guidewire is to be passed through; the inner rod is partially fixed to the guidewire insertion member; and the guidewire insertion member having a cross-sectional area in a cross-section perpendicular to an axis direction satisfying the formula (1):

cross-sectional area Sa>cross-sectional area Sb (1).

### TECHNICAL FIELD

The present invention relates to a delivery device for a medical tubular body.

### BACKGROUND ART

In recent years, a minimum invasive therapy in which a medical tubular body is delivered to a lesion site in the body has been developed. In the therapy, a delivery device is used that delivers a medical tubular body through body lumens and places it at a lesion site. The delivery device has an outer tube that holds the medical tubular body therein, and delivers the medical tubular body to a lesion site through body lumens, keeping the medical tubular body to be held in the outer tube. After delivering the medical tubular body, the medical tubular body can be released from the inside of the outer tube to be placed on the lesion site.

The medical tubular body includes, for example, a stent, a stent graft, an obturator, an infusion catheter, and a prosthesis valve. Of these, the stent is generally a medical tubular body used for treating various diseases caused by stenosis or occlusion of blood vessels or other body lumens.

It is required for a delivery device delivering the above medical tubular body to have good trackability and handleability, which means that handling of an operator is easy to be transferred to a distal side, such that the delivery device can be smoothly passed through body lumens, and should be unlikely to be deformed, kinked, or broken. A delivery device for a medical tubular body is, for example, disclosed in Patent Document 1. The Patent Document 1 discloses a stent delivery catheter including an outer shaft and an inner shaft; and the outer shaft having dual lumens, the inner shaft having a guide tube for a guidewire and a pusher wire in parallel, wherein the guide tube for a guidewire and the pusher wire are separately and slidably extended in the dual lumens of the outer shaft, and the pusher wire is connected to a midway in the longitudinal direction of the guide tube for a guidewire.

### RELATED ART DOCUMENT

### Patent Document

Patent Document 1: JP 5664040 B2

### SUMMARY OF THE INVENTION

### Problems to be Solved by the Invention

An example of an embodiment of a conventional delivery device for a medical tubular body is shown in FIG. 1. FIG. 1 is a cross-sectional schematic view of a conventional delivery device for a medical tubular body having an outer tube 12 in which a medical tubular body 11 is placed, an inner rod 13 disposed in the outer tube 12, and a guidewire insertion member 14 placed proximal to the medical tubular body 11, and a part of the inner rod 13 is fixed to the guidewire insertion member 14. The inner rod 13 extends to a proximal end of the delivery device for a medical tubular body (the side of an operator's hands), and an operator's handling can be transferred to a distal side through the inner rod 13. By pulling the outer tube 12 to a proximal side, the medical tubular body 11 is pushed out from the inside of the outer tube 12 to body lumens by the guidewire insertion member 14, and released. In particular, pulling the outer tube 12 makes the medical tubular body 11 move to a proximal side as the outer tube 12 moves to the proximal side, and the medical tubular body 11 compresses the guidewire insertion member 14 and the inner rod 13. It is not until reactive force generated in the guidewire insertion member 14 and the inner rod 13 against the compression stress surpasses friction resistance of the medical tubular body 11 that the medical tubular body 11 is released to the outside of the outer tube 12. However, it is known that the load to pull the outer tube 12 to the proximal side should be greater in the conditions where the friction resistance of the medical tubular body 11 is excessively large, the guidewire insertion member 14 and the inner rod 13 is too flexible, and these components have a structure preventing the load from being transferred in the longitudinal direction.

As shown in FIG. 1, the guidewire insertion member 14 has a penetration passage 15 in which a guidewire is to be passed through, and a guidewire tube 16 fixed to the penetration passage 15 is disposed distal to the penetration passage 15. A distal end of the guidewire tube 16 extends to the inside of a tip 17. Since a delivery device for a medical tubular body having such a configuration is inserted into body lumens, which are tortuous, the delivery device for a medical tubular body should be curved in accordance with the shape of the body lumens. However, local stress concentration at the proximal side of the guidewire insertion member 14 causes excessive bent of the device, and it is liable to kink. In addition, if the outer tube 12 is pulled to the proximal side while the device is kinked, it may be difficult for the medical tubular body 11 to be pushed out to body lumens. To make it harder for the delivery device for a medical tubular body to kink, for example, one option is to improve strength of the outer tube 12, however, its improved strength may cause poor trackability because the device becomes difficult to change its shape in accordance with the shape of body lumens when it passes through tortuous part or bifurcation part of the body lumens. As a result, contact resistance between the delivery device for a medical tubular body and the body lumen increases, and it is considered that the device becomes difficult to be inserted into the body lumen, resulting in decreased handleability.

The present invention has been achieved focusing on the circumstances described above, and the purpose thereof is to offer a delivery device for a medical tubular body that is less likely to kink without decreasing handleability.

### Means for Solving the Problems

The present invention includes the following inventions.

[1] A delivery device for a medical tubular body, comprising: an outer tube in which the medical tubular body is to be disposed; an inner rod disposed in the outer tube; and a guidewire insertion member disposed proximal to the medical tubular body, the guidewire insertion member having a penetration passage in which a guidewire is to be passed through; the inner rod partially fixed to the guidewire insertion member; and the guidewire insertion member having a cross-sectional area in a cross-section perpendicular to an axis direction satisfying the following formula (1).

cross-sectional area Sa>cross-sectional area Sb (1)

in the formula (1), the cross-sectional area Sa is a cross-sectional area of the guidewire insertion member at a center position in the axis direction between a distal-most position of the guidewire insertion member and a proximal-most position of a fixing region where the guidewire insertion member and the inner rod are fixed to each other, and in the formula (1), the cross-sectional area Sb is a cross-sectional area of the guidewire insertion member at a proximal-most position of the fixing region.

[2] The delivery device for a medical tubular body according to [1], wherein given that axis line (a) is an axis line through a gravity center of the penetration passage, axis line (b) is an axis line through a gravity center of the guidewire insertion member in a cross-section perpendicular to the axis direction at a center position in the axis direction between a distal-most position of the guidewire insertion member and a proximal-most position of the fixing region of the guidewire insertion member, and axis line (c) is an axis line through a gravity center of the inner rod, distance (a-c) between the axis line (a) and the axis line (c) is longer than distance (a-b) between the axis line (a) and the axis line (b).

[3] The delivery device for a medical tubular body according to [1] or [2], wherein a proximal end part of the guidewire insertion member has a taper shape.

[4] The delivery device for a medical tubular body according to any one of [1] to [3], further comprising a protection member disposed outside the inner rod at a proximal side to the fixing region

[5] The delivery device for a medical tubular body according to [4], wherein a distance between a distal-most position of the protection member and a proximal-most position of the guidewire insertion member is 30 mm or less.

[6] The delivery device for a medical tubular body according to any one of [1] to [5], wherein the delivery device is of a rapid exchange type where the outer tube has a guidewire port, and the guidewire port is disposed distal to a proximal end of the outer tube.

[7] The delivery device for a medical tubular body according to any one of [1] to [6], further comprising a guidewire tube disposed in the penetration passage, wherein the guidewire is to be passed through in the guidewire tube; and the guidewire tube extends from a side that is proximal to a proximal opening of the penetration passage to a side that is distal to a distal opening of the penetration passage.

[8] The delivery device for a medical tubular body according to any one of [1] to [6], further comprising an inner tube disposed in the outer tube, wherein the guidewire is to be passed through in the inner tube; and a distal end of the inner tube and a proximal opening of the penetration passage are fixed to each other.

[9] The delivery device for a medical tubular body according to any one of [1] to [6], and [8], further comprising a guidewire tube disposed in the outer tube, wherein the guidewire is to be passed through in the guidewire tube; and a proximal end of the guidewire tube and a distal opening of the penetration passage are fixed to each other.

[10] The delivery device for a medical tubular body according to [6], further comprising an internal tube disposed in the outer tube, wherein the guidewire is to be passed through in the internal tube; and a proximal end of the internal tube is fixed to the guidewire port.

[11] The delivery device for a medical tubular body according to [10], wherein a part of the internal tube is disposed in the penetration passage.

[12] The delivery device for a medical tubular body according to [10] or [11], further comprising a guidewire tube disposed in the penetration passage, wherein the guidewire is to be passed through in the guidewire tube.

[13] The delivery device for a medical tubular body according to [10] or [11], further comprising a guidewire tube disposed in the outer tube, wherein the guidewire is to be passed through in the guidewire tube; and a proximal end of the guidewire tube and a distal opening of the penetration passage are fixed to each other.

[14] The delivery device for a medical tubular body according to [12], wherein a part of the internal tube is disposed in the guidewire tube.

[15] The delivery device for a medical tubular body according to [12] or [14], wherein a proximal end of the guidewire tube is located proximal to a proximal opening of the penetration passage.

[16] The delivery device for a medical tubular body according to any one of [12], [14], and [15], wherein a proximal end part of the guidewire tube has a taper shape.

[17] The delivery device for a medical tubular body according to any one of [1] to [16], wherein the guidewire insertion member is colored.

[18] The delivery device for a medical tubular body according to any one of [1] to [17], wherein the medical tubular body is a self-expanding stent.

### Effects of the Invention

According to the present invention, a delivery device for a medical tubular body can be offered that is less likely to kink without decreasing handleability even though it is excessively bent, because a guidewire insertion member is formed such that a cross-sectional area of the guidewire insertion member in a cross-section perpendicular to an axis direction satisfies a certain relationship.

### BRIEF DESCRIPTION OF THE DRAWINGS

FIG. 1 is a cross-sectional view showing an example of an embodiment of a conventional delivery device for a medical tubular body.

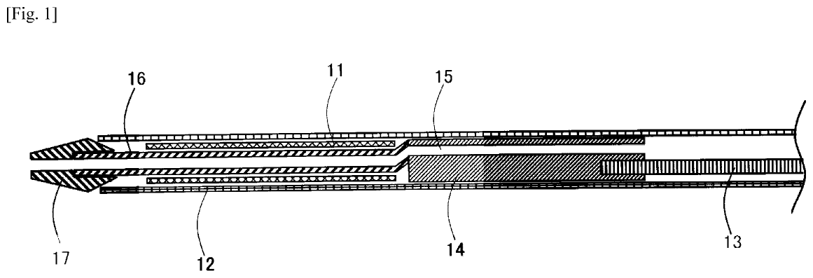

FIG. 2 is a cross-sectional view showing an example of an embodiment according to the present invention.

FIG. 3 is a cross-sectional view showing a positional relationship of a penetration passage formed in a guidewire insertion member and an inner rod fixed to the guidewire insertion member.

FIG. 4 is a cross-sectional view showing another example of an embodiment according to the present invention.

FIG. 5 is a cross sectional view showing another example of an embodiment according to the present invention.

FIG. 6 is a cross sectional view showing another example of an embodiment according to the present invention.

FIG. 7 is a cross sectional view showing another example of an embodiment according to the present invention.

FIG. 8 is a cross sectional view showing another example of an embodiment according to the present invention.

FIG. 9 is a cross sectional view showing another example of an embodiment according to the present invention.

### MODE FOR CARRYING OUT THE INVENTION

A delivery device for a medical tubular body according to the present invention has an outer tube in which the medical tubular body is disposed, an inner rod disposed in the outer tube, and a guidewire insertion member disposed proximal to the medical tubular body; and the guidewire insertion member has a penetration passage in which a guidewire is to be passed through, the inner rod is partially fixed to the guidewire insertion member, and a cross-sectional area of the guidewire insertion member in a cross-section perpendicular to an axis direction satisfies the following formula (1).

cross-sectional area Sa>cross-sectional area Sb (1)

In the formula (1), the cross-sectional area Sa is a cross-sectional area of the guidewire insertion member at a center position in an axis direction between a distal-most position of the guidewire insertion member and a proximal-most position of a fixing region where the guidewire insertion member and the inner rod is fixed, and in the formula (1), the cross-sectional area Sb is a cross-sectional area of the guidewire insertion member at a proximal-most position of the fixing region.

Since forming the guidewire insertion member such that the cross-sectional area of the guidewire insertion member in a cross-section perpendicular to the axis direction satisfies the relationship shown in the above formula (1) makes a cross-sectional area of the guidewire insertion member at a proximal side smaller, it becomes easier for stress to keep from locally concentrating when the delivery device for a medical tubular body is passed through tortuous body lumens. In other words, the stiffness in the axis direction of the delivery device for a medical tubular body gradually changes, which makes it unlikely for the delivery device for a medical tubular body to kink. In addition, since the guidewire insertion member has smaller cross-section area at the proximal-most position of a fixing region to the inner rod, stress imposed on the guidewire insertion member from the medical tubular body when the medical tubular body is released can be concentrated on the inner rod. Accordingly, it becomes easier for unnecessary force not to be generated when the outer tube is pulled to the proximal side to release the medical tubular body, which can reduce risks including breaking of the outer tube or delivery of the medical tubular body to incorrect position off the expected position, and as a result, safer treatment is available.

Hereinafter, an example of an embodiments of a delivery device for a medical tubular body according to the present invention will be described in reference to figures, however, the present invention is not limited by the figures, and can be altered in design within a scope in compliance with the intent described above and below, and all the changes are to be encompassed within a technical scope of the present invention.

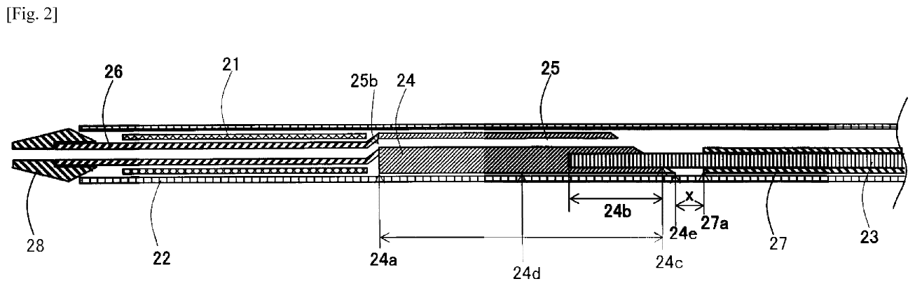

FIG. 2 is a cross-sectional schematic view showing an example of an embodiment of a delivery device for a medical tubular body according to the present invention. Note that FIG. 2 only includes a distal part of the delivery device for a medical tubular body.

The delivery device for a medical tubular body has an outer tube 22 in which a medical tubular body 21 is disposed, an inner rod 23 disposed in the outer tube 22, and a guidewire insertion member 24 located proximal to the medical tubular body 21.

The guidewire insertion member 24 has a penetration passage 25 in which a guidewire is to be passed through, and the inner rod 23 is partially fixed to the guidewire insertion member 24. Any method for fixing the inner rod 23 to the guidewire insertion member 24 is available, and for example, it includes a method in which the inner rod 23 is press fitted into the guidewire insertion member 24 to be fixed (being pinched), or a method in which the inner rod 23 is inserted into a lumen that is preliminarily formed in the guidewire insertion member 24 and the inner rod 23 and the lumen are fixed together with an adhesive or the like.

The guidewire insertion member 24 has a cross-section area in a cross section perpendicular to an axis direction of the guidewire insertion member 24 satisfying the following formula (1).

cross-sectional area Sa>cross-sectional area Sb (1)

In the above formula (1), the cross-sectional area Sa is a cross-sectional area of the guidewire insertion member 24 at a center position 24d in an axis direction between a distal-most position 24a of the guidewire insertion member 24 and a proximal-most position 24c of a fixing region 24b where the guidewire insertion member 24 and the inner rod 23 are fixed to each other. The cross-sectional area Sa is an area calculated on the basis of an outline of the guidewire insertion member 24 at the center position 24d, which should be calculated including an area of the penetration passage 25.

In the above the formula (1), the cross-sectional area Sb is a cross-sectional area of the guidewire insertion member 24 at a proximal-most position 24c of the fixing region 24b of the guidewire insertion member 24. The cross-sectional area Sb is an area calculated on the basis of an outline of the guidewire insertion member 24 at the proximal-most position 24c.

Since forming the guidewire insertion member such that the cross-sectional area in a cross-section perpendicular to the axis direction of the guidewire insertion member satisfies the relationship shown in the above formula (1) makes a cross-sectional area of the guidewire insertion member at a proximal side smaller, it becomes easier for stress to keep from locally concentrating when the delivery device for a medical tubular body is passed through tortuous body lumens. In other words, the stiffness in the axis direction of the delivery device for a medical tubular body gradually changes, which makes it unlikely for the delivery device for a medical tubular body to kink. In addition, since the guidewire insertion member has smaller cross-sectional area at the proximal-most position of the fixing region to the inner rod, stress imposed on the guidewire insertion member from the medical tubular body when the medical tubular body is released can be concentrated on the inner rod. Accordingly, it becomes easier for unnecessary force not to be generated when the outer tube is pulled to the proximal side to release the medical tubular body, which can reduce risks including breaking of the outer tube or delivery of the medical tubular body to incorrect position off the expected position, and as a result, safer treatment is available.

In the outer tube 22, a guidewire tube 26 in which the guidewire is passed through is disposed, and a proximal end of the guidewire tube 26 just should be, for example shown in FIG. 2, fixed to a distal opening 25b of the penetration passage 25. Thanks to the penetration passage 25 and the guidewire tube 26 that are in communication with each other, the guidewire can be passed through the inside of them, and therefore, the delivery device for a medical tubular body can be easily inserted into body lumens guided by the inserted guidewire. The distal opening 25b of the penetration passage 25 and the guidewire tube 26 are fixed to each other by, for example, thermal fusion bonding and the like. In FIG. 2, a distal end of the guidewire tube 26 extends to the inside of a tip 28.

The present invention is not limited to the configuration where the proximal end of the guidewire tube 26 is fixed to the distal opening 25b of the penetration passage 25, and as described later, may include the configuration where the guidewire tube 26 is disposed in the penetration passage 25, and the guidewire tube 26 extends from a side that is proximal to a proximal opening of the penetration passage 25 to a side that is distal to the distal opening 25b of the penetration passage 25.

In addition, at a distal end of the guidewire insertion member 24, a pusher that makes it easier for the medical tubular body 21 to be pushed out may be disposed.

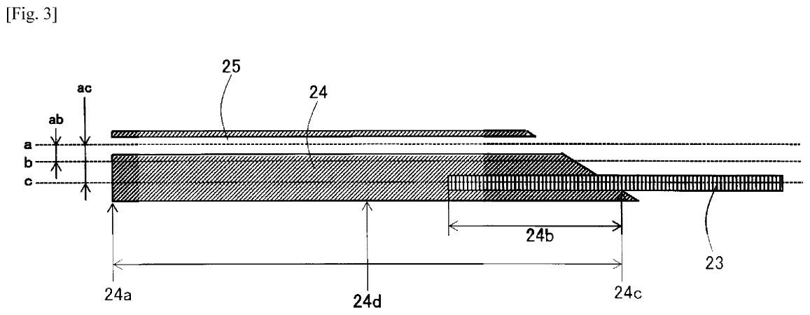

The guidewire insertion member 24 is preferably configured such that, as shown in FIG. 3, given that an axis line (a) is an axis line through a gravity center of the penetration passage 25; an axis (b) is an axis line through a gravity center of the guidewire insertion member 24 in a cross-section perpendicular to the axis direction at a center position 24d in the axis direction between a distal-most position 24a of the guidewire insertion member 24 and a proximal-most position 24c of the fixing region 24b of the guidewire insertion member24; and an axis line (c) is an axis line through a gravity center of the inner rod 23, distance (a-c) between the axis line (a) and the axis line (c) is preferably longer than distance (a-b) between the axis line (a) and the axis line (b). The axis lines (a) to (c) satisfying the above relationship can make stress imposed on the guidewire insertion member from the medical tubular body when the medical tubular body is released to be concentrated on the inner rod. Accordingly, it becomes easier for unnecessary force not to be generated when the outer tube is pulled to the proximal side to release the medical tubular body, which can reduce risks including breaking of the outer tube or delivery of the medical tubular body to incorrect position off the expected position, and as a result, safer treatment is available.

Any shape of the proximal end part of the guidewire insertion member 24 is available, as long as the cross-sectional area Sb of the guidewire insertion member 24 at the proximal-most position 24c of the fixing region 24b satisfies the relationship of the above formula (1), and the shapes are exemplified by a taper shape, a concavo-convex shape, a stepwise shape, and a wave shape. Of these, the taper shape is more preferable.

The guidewire insertion member 24 is preferably colored. Colored guidewire insertion member 24 makes it easier for it to be visually observed endoscopically, which makes it easier for the guidewire insertion member 24 to be located. The colored guidewire insertion member 24 is sometimes referred to as a visual marker. Any color is available for the guidewire insertion member 24 that is easy to be visually observed endoscopically, and for example, comparatively contrasting color to gastrointestinal mucous membrane or blood is preferable, and especially preferably yellow.

The inner rod 23, as shown in FIG. 2, may have a protection member 27 disposed outside the inner rod 23 at a proximal side of the fixing region 24b. The protection member 27 can prevent or reduce damage of the inner rod 23. In addition, the protection member 27 can improve pushability in the longitudinal direction of the inner rod 23, which improves force for supporting stress imposed on the inner rod 23 when the medical tubular body is released. Accordingly, the medical tubular body can be easily released.

Distance x between distal-most position 27a of the protection member 27 and a proximal-most position 24e of the guidewire insertion member 24 is preferably 30 mm or less. The distance x of 30 mm or less makes the stiffness of the delivery device for a medical tubular body in the longitudinal direction continuous, which improves resistance against bending of the device, and the device is less likely to kink. The distance x is preferably as short as possible. The distance x is more preferably 20 mm or less, even more preferably 10 mm or less, and especially preferably 5 mm or less. Thanks to the distance of 5 mm or less, the protection member 27 can improve pushability of the inner rod 23, and the protection member 27 can integrally support stress imposed from the guidewire insertion member along with the inner rod 23 when the medical tubular body is released.

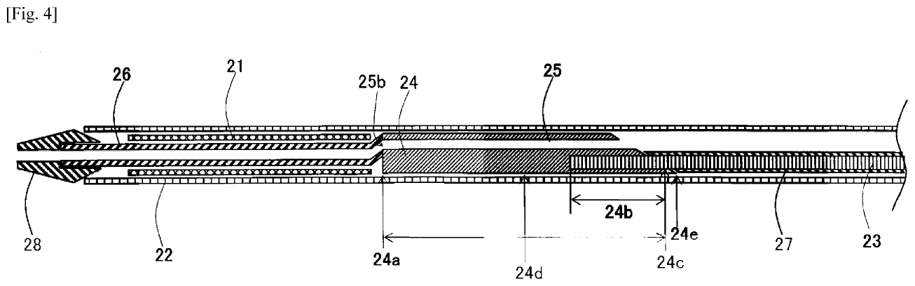

Any shape of a distal end part of the protection member 27 is available, and for example as shown in FIG. 4, the complementary shape to the shape of the proximal end part of the guidewire insertion member 24 is preferable. In FIG. 4, the same parts as in the above figures have the same reference signs to omit repeated explanation.

The complementary shape of the protection member 27 means that the protection member 27 has plane contact with the guidewire insertion member 24 when the distal end part of the protection member 27 and the proximal end part of the guidewire insertion member 24 are contact with each other. The complementary shape can reduce uncovered area of the inner rod 23, which can improve pushability of the inner rod 23 with the protection member 27, and the protection member 27 can integrally support stress imposed from the guidewire insertion member along with the inner rod 23 when the medical tubular body is released. Moreover, the complementary shape increases contact area of the protection member 27 with the guidewire insertion member 24, which can improve efficiency of stress transfer. The distal end part of the protection member 27 preferably has a taper shape, and the guidewire insertion member 24 also preferably has a taper shape.

Constituent materials of the protection member 27 are exemplified by resin materials including polyethylene, fluorine resin (such as PTFE, PFA and the like), polyamide, polyamide-based elastomer, polyurethane, polyester, silicone, and polyetheretherketone (PEEK).

The delivery device for a medical tubular body is preferably of rapid exchange type where the outer tube 22 has a guidewire port, and the guidewire port is preferably disposed distal to a proximal end of the outer tube 22.

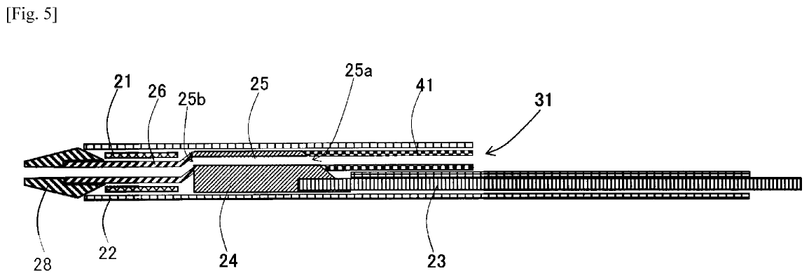

The delivery device for a medical tubular body may be configured, as shown in FIG. 5, such that inner tube 41 in which the guidewire is to be passed through is disposed in the outer tube 22, and a distal end of the inner tube 41 and the proximal opening 25a of the penetration passage 25 may be fixed to each other. Note that in FIG. 5 the reference sign 31 shows the position of the guidewire port. In FIG. 5, the same parts as in the above figures have the same reference signs to omit repeated explanation.

Methods for fixing the distal end of the inner tube 41 and the proximal opening 25a of the penetration passage 25 to each other are exemplified by a method including preparing the guidewire insertion member 24 and the inner tube 41, and fixing them together by thermal fusion bonding or adhesive bonding.

While the rapid exchange type is shown as an embodiment of the delivery device for a medical tubular body in FIG. 5, a device of over-the-wire type is also available.

The delivery device for a medical tubular body shown in FIG. 5 has the configuration where the guidewire tube 26 in which the guidewire is to be passed through is disposed in the outer tube 22, and the proximal end of the guidewire tube 26 and the distal opening 25b of the penetration passage 25 are fixed to each other. The distal opening 25b of the penetration passage 25 and the guidewire tube 26 may be fixed together, for example, by thermal fusion bonding and the like.

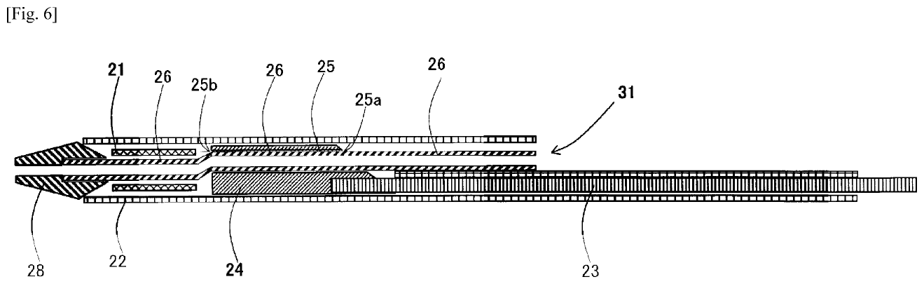

The delivery device for a medical tubular body has the configuration, as shown in FIG. 6, where the guidewire tube 26 in which the guidewire is to be passed through is disposed in the penetration passage 25, and the guidewire tube 26 may extends from the side that is proximal to the proximal opening 25a of the penetrating tract 25 to the side that is distal to the distal opening 25b of the penetration passage 25. In FIG. 6, the same parts as in the above figures have the same reference signs to omit repeated explanation.

While the rapid exchange type is shown as an embodiment of the delivery device for a medical tubular body in FIG. 6, a device of over-the-wire type is also available.

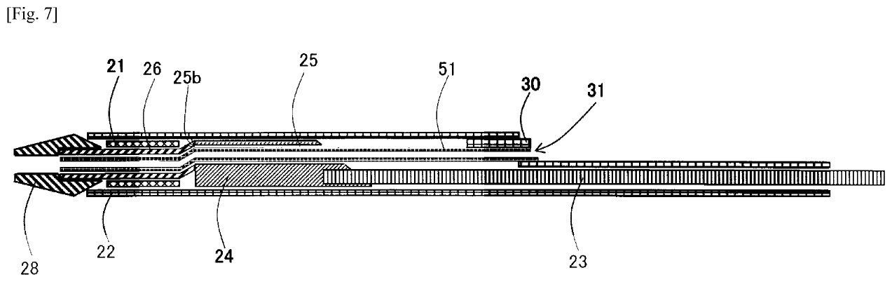

The delivery device for a medical tubular body has the configuration, as shown in FIG. 7, where internal tube 51 in which the guidewire is passed through is disposed in the outer tube 22, and a proximal end of the internal tube 51 may be fixed to the guidewire port 31. In FIG. 7, the same parts as in the above figures have the same reference signs to omit repeated explanation.

As shown in FIG. 7, the proximal end part of the internal tube 51 and the outer tube 22 are pressed to each other to be fixed with a filler 30 disposed between the internal tube 51 and the outer tube 22.

Methods for fixing the internal tube 51 are exemplified by a method where the filler 30 is formed by thickening a part of the outer tube 22 to fill a gap with the proximal end part of the internal tube 51, a method where the filler 30 composed of resin and the like is disposed between the proximal end part of the internal tube 51 and the outer tube 22 and they are fixed together with adhesive, or a method where the proximal end part of the internal tube 51 and the outer tube 22 is adhesive bonded with the filler 30 that is used as an adhesive. Alternatively, an inner wall of the outer tube 22 and an outer wall of the internal tube 51 may be pressed to each other to be fixed without the filler 30, for example, by thermal fusion bonding or pressure bonding.

The embodiment of the delivery device for a medical tubular body shown in FIG. 7 is preferably of rapid exchange type.

A part of the internal tube 51 may be disposed in the penetration passage 25 as shown in FIG. 7. Such a configuration can prevent the guidewire from tangling with the inner rod 23 and the like, which may cause the inner rod 23 impossible to be operated, or can prevent the guidewire from precluding the release of the medical tubular body when the medical tubular body is pushed out and released by pulling the outer tube 22 to the proximal side, because the guidewire is necessarily disposed inside the internal tube 51 or the penetration passage 25.

The delivery device for a medical tubular body shown in the FIG. 7 has the configuration where the guidewire tube 26 in which the guidewire is to be passed through is disposed in the outer tube 22, and the proximal end of the guidewire tube 26 and the distal opening 25b of the penetration passage 25 are fixed to each other. The distal opening 25b and the guidewire tube 26 may be fixed together, for example, by thermal fusion bonding and the like.

The internal tube 51 may be extended to the distal side of the penetration passage 25 through the penetration passage 25, and disposed in the guidewire tube 26 fixed to the distal opening 25b of the penetration passage 25. Such a configuration can prevent the guidewire from tangling with the inner rod 23 and the like, which may cause the inner rod 23 impossible to be operated, or can prevent the guidewire from precluding the release of the medical tubular body when the medical tubular body is pushed out and released by pulling the outer tube 22 to the proximal side, because the guidewire is necessarily disposed inside the internal tube 51, the penetration passage 25, or the guidewire tube 26.

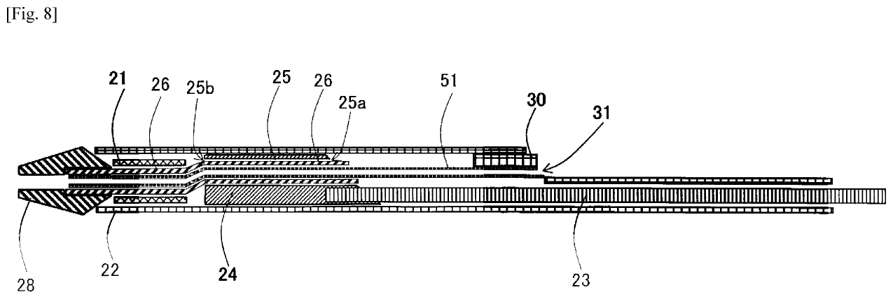

The delivery device for a medical tubular body may have the configuration, as shown in FIG. 8, where the guidewire tube 26 in which the guidewire is to be passed through is disposed in the penetration passage 25. In FIG. 8, the same parts as in the above figures have the same reference signs to omit repeated explanation.

A part of the internal tube 51 may be disposed in the guidewire tube 26 as shown in FIG. 8. Such a configuration can prevent the guidewire from tangling with the inner rod 23 and the like, which may cause the inner rod 23 impossible to be operated, or can prevent the guidewire from precluding the release of the medical tubular body when the medical tubular body is pushed out and released by pulling the outer tube 22 to the proximal side, because the guidewire is necessarily disposed inside the internal tube 51 or the guidewire tube 26.

The proximal end of the guidewire tube 26 may be located on the side that is proximal to the proximal opening 25a of the penetration passage 25, as shown in FIG. 8. Such a configuration makes it easier for the internal tube 51 to be disposed in the guidewire tube 26.

The shape of the proximal end part of the guidewire tube 26 is exemplified by a taper shape, a concavo-convex shape, a stepwise shape, and a wave shape, and of these, the taper shape is more preferable.

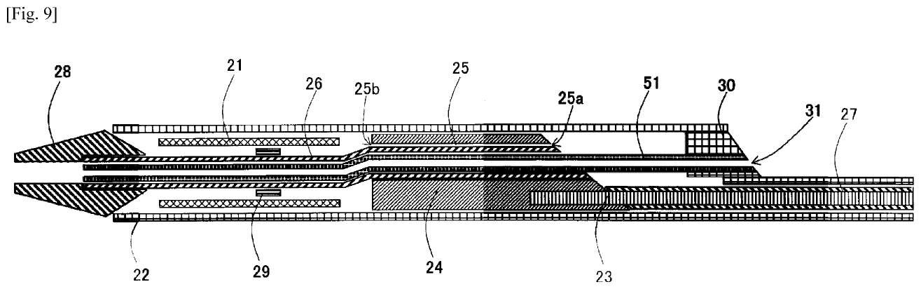

Another example of embodiments of the delivery device for a medical tubular body according to the present invention is shown in FIG. 9. In FIG. 9, the same parts as in the above figures have the same reference signs to omit repeated explanation.

The delivery device for a medical tubular body shown in FIG. 9 has the configuration where the distal end part of the protection member 27 and the proximal end part of the guidewire insertion member 24 are made to have a complementary shape.

In the configuration, the guidewire tube 26 in which the guidewire is to be passed through is disposed in the penetration passage 25, the distal side of the guidewire tube 26 extends to the tip 28, the proximal side of the guidewire tube 26 extends to the position of the proximal opening 25a of the penetration passage 25. The shape of the proximal end part of the guidewire tube 26 corresponds to the shape of the proximal end part of the guidewire insertion member 24. The shape of the proximal end part of the guidewire tube 26 corresponding to the shape of the proximal end part of the guidewire insertion member 24 makes it easier for the internal tube 51 to be inserted into the guidewire tube 26. Note that corresponding shape means that the proximal opening of the guidewire tube 26 and the proximal opening of the guidewire insertion member 24 are in the same plane when seeing the proximal opening of the guidewire tube 26 from the proximal side in the longitudinal direction. In another words, the proximal end face of the guidewire insertion member 24 and the proximal end face of the guidewire tube 26 are in the same plane.

To make the shape of the proximal end part of the guidewire tube 26 and the shape of the proximal end part of the guidewire insertion member 24 corresponding to each other, for example, the guidewire insertion member 24 and the guidewire tube 26 may be cut off together in the condition where the guidewire tube 26 is inserted in the guidewire insertion member 24.

In the configuration shown in FIG. 9, the internal tube 51 in which the guidewire is to be passed through is disposed in the outer tube 22, the proximal end of the internal tube 51 is fixed to the guidewire port 31, and the internal tube 51 extends to the tip 28 through the inside of the guidewire tube 26 disposed in the penetration passage 25.

In the FIG. 9, the filler 30 is disposed between the internal tube 51 and the outer tube 22. The shape of the proximal end part of the filler 30 corresponds to the shape of the proximal end part of the internal tube 51. The shape of the proximal end part of the filler 30 and the shape of the proximal end part of the internal tube 51 corresponding to each other makes it easier for the guidewire to be inserted into the internal tube 51.

As shown in FIG. 9, locking part 29 may be disposed on the outside of the guidewire tube 26. The locking part 29 engages an inner surface of the medical tubular body 21, which prevents the medical tubular body 21 from going backward by the locking part 29 when the outer tube 22 is pulled to the proximal side, and the medical tubular body 21 is released outwardly.

The locking part 29 may be disposed at any position as long as the locking part 29 can prevent the medical tubular body 21 from going backward, and for example, it is disposed at a position proximal to the center position of a longitudinal length of the medical tubular body 21.

Alternatively, without the locking part 29, the proximal end of the medical tubular body 21 may be supported by the distal end of the guidewire insertion member 24.

The medical tubular body includes, for example, a stent, a stent graft, an obturator, an infusion catheter, and a prosthesis valve.

Of these, a stent is preferable. The stent can be exemplified by a stent having a coiled shape formed with one wire made of metal or polymer material, a stent obtained by cutting with a laser from a metal tube, a stent obtained by assembling linear materials by laser welding, or a stent obtained by weaving more than one metal wire. The stent is classified into a balloon-expandable stent having a stent that expands with a mounted balloon, and a self-expanding stent that expands by itself by removing an restrainer of expansion of the stent. In the present invention, the self-expanding stent is preferably used.

The present application claims priority based on Japanese Patent Application No. 2018-157665 filed on Aug. 24, 2018. All the contents described in Japanese Patent Application No. 2018-157665 filed on Aug. 24, 2018 are incorporated herein by reference.

### DESCRIPTION OF REFERENCE SIGNS

11: medical tubular body
12: outer tube
13: inner rod
14: guidewire insertion member
15: penetration passage
16: guidewire tube
17: tip
21: medical tubular body
22: outer tube
23: inner rod
24: guidewire insertion member
24a: distal-most position of guidewire insertion member 24
24b: fixing region where guidewire insertion member 24 and inner rod 23 are fixed to each other
24c: proximal-most position of fixing region where guidewire insertion member 24 and inner rod 23 are fixed to each other
24d: center position in axis direction between distal-most position 24a of guidewire insertion member 24 and proximal-most position 24c of fixing region 24b where guidewire insertion member 24 and inner rod 23
25: penetration passage
25a: proximal opening of penetration passage 25
25b: distal opening of penetration passage 25
26: guidewire tube
27: protection member
28: tip
29: locking part
30: filler
31: guidewire port
41: inner tube
51: internal tube

1. A delivery device for a medical tubular body, comprising:
an outer tube in which the medical tubular body is to be disposed, the outer tube having a distal side and a proximal side;
an inner rod disposed in the outer tube; and
a guidewire insertion member disposed proximal to the medical tubular body, the guidewire insertion member having a penetration passage, through which a guidewire is to be passed;
the inner rod partially fixed to the guidewire insertion member; and
the guidewire insertion member having a cross-sectional area in a cross-section perpendicular to an axis direction satisfying the following formula (1):
cross-sectional area Sa>cross-sectional area Sb (1),
in the formula (1), the cross-sectional area Sa is a cross-sectional area of the guidewire insertion member at a middle portion in the axis direction between a distal end of the guidewire insertion member and a proximal end of a fixing portion where the guidewire insertion member and the inner rod are fixed to each other, and the cross-sectional area Sb is a cross-sectional area of the guidewire insertion member at the proximal end of the fixing portion.

2. The delivery device for a medical tubular body according to claim 1, wherein the outer tube, the inner rod, and the guidewire insertion member are arranged such that a distance (a-c) between an axis line (a) and an axis line (c) is longer than a distance (a-b) between the axis line (a) and an axis line (b),
given that the axis line (a) is an axis line passing through a gravity center of a cross-section of the penetration passage, the axis line (b) is an axis line passing through a gravity center of a cross-sectional shape defined by an outer circumferential line of the guidewire insertion member at the middle portion in the axis direction between the distal end of the guidewire insertion member and the proximal end of the fixing portion of the guidewire insertion member, and the axis line (c) is an axis line through a gravity center of a cross-section of the inner rod, each of the cross-sections perpendicular to the axis direction.

3. The delivery device for a medical tubular body according to claim 1, wherein the guidewire insertion member has a taper shape at the proximal end.

4. The delivery device for a medical tubular body according to claim 1, further comprising a protection member disposed outside the inner rod, inside the outer tube, and at a more proximal side than the fixing portion.

5. The delivery device for a medical tubular body according to claim 4, wherein a distance between a distal end of the protection member and a proximal end of the guidewire insertion member is 30 mm or less.

6. The delivery device for a medical tubular body according to claim 1, wherein the outer tube has a guidewire port, and the guidewire port is disposed at a more distal side than a proximal end of the outer tube.

7. The delivery device for a medical tubular body according to claim 1, further comprising a guidewire tube, through which the guidewire is to be passed, wherein
the guidewire tube is disposed in the penetration passage of the guidewire insertion member such that the guidewire tube extends from a side that is proximal to a proximal opening of the penetration passage to a side that is distal to a distal of the penetration passage.

8. The delivery device for a medical tubular body according to claim 1, further comprising an inner tube disposed in the outer tube, wherein
the guidewire is to be passed through in the inner tube; and
a distal end of the inner tube is connected to a proximal opening of the penetration passage.

9. The delivery device for a medical tubular body according to claim 1, further comprising a guidewire tube disposed in the outer tube, wherein
the guidewire is to be passed through in the guidewire tube; and
a proximal end of the guidewire tube is connected to a distal opening of the penetration passage.

10. The delivery device for a medical tubular body according to claim 6, further comprising an internal tube disposed in the outer tube, wherein
the guidewire is to be passed through in the internal tube; and
a proximal end of the internal tube is connected to the guidewire port.

11. The delivery device for a medical tubular body according to claim 10, wherein a part of the internal tube is disposed in the penetration passage.

12. The delivery device for a medical tubular body according to claim 10, further comprising a guidewire tube disposed in the penetration passage, wherein the guidewire is to be passed through in the guidewire tube.

13. The delivery device for a medical tubular body according to claim 10, further comprising a guidewire tube disposed in the outer tube, wherein
the guidewire is to be passed through in the guidewire tube; and
a proximal end of the guidewire tube is connected to a distal opening of the penetration passage.

14. The delivery device for a medical tubular body according to claim 12, wherein a part of the internal tube is disposed in the guidewire tube.

15. The delivery device for a medical tubular body according to claim 12, wherein the guidewire tube and the guidewire insertion member are arranged such that the guidewire tube extends from a proximal opening of the penetration passage toward a proximal side.

16. The delivery device for a medical tubular body according to claim 12, wherein a proximal end part of the guidewire tube has a taper shape.

17. The delivery device for a medical tubular body according to claim 1, wherein the guidewire insertion member is colored.

18. The delivery device for a medical tubular body according to claim 1, wherein the medical tubular body is a self-expanding stent.
